(19) United States
(12) Patent Application Publication
(10) Pub. No.: US 2025/0152377 A1
(43) Pub. Date: May 15, 2025

(54) CERVICAL INTERBODY

(71) Applicant: Evolution Spine, LLC, Dallas, TX (US)

(72) Inventors: Francia Sanchez Villasana, Knoxville, TN (US); James Mirda, Germantown, TN (US); Todd Wallenstein, Ashburn, VA (US); Benjamin Keller, Raleigh, NC (US); Keith Roby, Allen, TX (US)

(21) Appl. No.: 18/927,151

(22) Filed: Oct. 25, 2024

Related U.S. Application Data
(60) Provisional application No. 63/598,607, filed on Nov. 14, 2023.

(51) Int. Cl. A61F 2/44; A61F 2/30

(52) U.S. Cl. CPC A61F 2/4455; A61F 2/3094; A61F 2002/30484; A61F 2250/0058

(57) ABSTRACT

Provided herein is a spinal interbody device including an interbody; and a movable backout prevention mechanism disposed at least partially within the interbody, comprising a body and one or more lobes integral with the body, wherein each of the one or more lobes is configured to be moved between an unlocked position for insertion of a fixation member and a locked position for preventing backout of the fixation member.

### CROSS-REFERENCE TO RELATED APPLICATIONS

This application claims priority to U.S. Provisional Application Ser. No. 63/598,607, filed Nov. 14, 2023, the entire contents of which are incorporated herein by reference.

### STATEMENT OF FEDERALLY FUNDED RESEARCH

Not applicable.

### TECHNICAL FIELD OF THE INVENTION

The present invention relates in general to surgery on the human spine, and more particularly, to an interbody for use in interbody fusion.

### BACKGROUND OF THE INVENTION

Without limiting the scope of the invention, its background is described in connection with a cervical interbody as an example.

An intervertebral disc in a human spine may be replaced by a spinal interbody to promote fusion of the vertebrae adjacent to the disc. Current interbody devices that use screw or anchor fixation to secure an interbody to an upper or lower vertebral body currently rely on a secondary mechanism that is manufactured separately and then assembled into the interbody to prevent backout of the screw or anchor from the interbody device. These secondary mechanisms are susceptible to failure because they are separate devices that require assembly. The secondary assembly requirements of these backout prevention mechanisms limit the design intent of the interbody devices, increase the manufacturing costs, and require a design that accounts for potential failure due to separation of the mechanism form the interbody. Traditionally, these backout prevention mechanisms are threaded, swaged, welded, pinned, or use some other form of mechanical fixation onto the interbody. The drawbacks of these secondary mechanical fixation methods include an increase in the size of the backout prevention mechanism, an increase in manufacturing costs, increased manufacturing time, and additional components that may increase the potential of failure after implantation.

A need exists for an interbody with a movable backout prevention mechanism that is integrated to the interbody to reduce assembly requirements, reduce size requirements, and allows for new interbody designs that better integrate the mechanism, which also reduces the overall size of the mechanism and may allow for more room for bony ingrowth. An integrated movable backout prevention mechanism is also less likely to separate from the interbody, reducing risks to the patient.

### SUMMARY OF THE INVENTION

As embodied and broadly described herein, an aspect of the present disclosure relates to a spinal interbody device including an interbody; and a movable backout prevention mechanism disposed at least partially within the interbody, including a body and one or more lobes integral with the body, wherein each of the one or more lobes is configured to be moved between an unlocked position for insertion of a fixation member and a locked position for preventing backout of the fixation member. In one aspect, the backout prevention mechanism is configured to be rotatably moved between the unlocked position and the locked position. In another aspect, the backout prevention mechanism includes a detent configured to engage an indent to provide tactile feedback to a user and to prevent free rotation of the backout prevention mechanism. In another aspect, at least a portion of the backout prevention mechanism is disposed between an anterior face of an anterior wall of the interbody and a posterior face of the anterior wall. In another aspect, the body has a shape that is conical, cylindrical, hexagonal, spherical, hemispherical, polyhedral, pyramidal, rectangular, or leaf-shaped, or includes spirally stacked discs. In another aspect, the device is manufactured using an additive manufacturing, forging, casting, melting, or growing process. In another aspect, the device includes titanium, tantalum, stainless steel, cobalt chrome molybdenum, or a plastic.

As embodied and broadly described herein, an aspect of the present disclosure relates to a spinal interbody device including an interbody; and a movable backout prevention mechanism disposed at least partially within the interbody including a body and one or more lobes integral with the body, wherein each of the one or more lobes is configured to be moved between an unlocked position for insertion of a fixation member and a locked position for preventing backout of the fixation member; and one or more tools to manipulate the backout prevention mechanism. In one aspect, the backout prevention mechanism is configured to be rotatably moved between the unlocked position and the locked position. In another aspect, the backout prevention mechanism includes a detent configured to engage an indent to provide tactile feedback to a user and to prevent free rotation of the backout prevention mechanism. In another aspect, at least a portion of the backout prevention mechanism is disposed between an anterior face of an anterior wall of the interbody and a posterior face of the anterior wall. In another aspect, the body has a shape that is conical, cylindrical, hexagonal, spherical, hemispherical, polyhedral, pyramidal, rectangular, or leaf-shaped, or includes spirally stacked discs. In another aspect, the device is manufactured using an additive manufacturing, forging, casting, melting, or growing process. In another aspect, the device includes titanium, tantalum, stainless steel, cobalt chrome molybdenum, or a plastic.

As embodied and broadly described herein, an aspect of the present disclosure relates to a method of using a spinal interbody device including providing a patient in need of implantation of a spinal interbody; providing the spinal interbody device, including an interbody; and a movable backout prevention mechanism disposed at least partially within the interbody, including a body and one or more lobes integral with the body, wherein each of the one or more lobes is configured to be moved between an unlocked position for insertion of a fixation member and a locked position for preventing backout of the fixation member; and implanting the spinal interbody device in the patient. In one aspect, the one or more lobes are each configured to be rotatably moved between the unlocked position and the locked position. In another aspect, the backout prevention mechanism includes a detent configured to engage an indent to provide tactile feedback to a user and to prevent free rotation of the backout prevention mechanism. In another aspect, at least a portion of the backout prevention mechanism is disposed between an anterior face of an anterior wall of the interbody and a posterior face of the anterior wall. In another aspect, the body has a shape that is conical, cylindrical, hexagonal, spherical, hemispherical, polyhedral, pyramidal, rectangular, or leaf-shaped, or includes spirally stacked discs. In another aspect, the device is manufactured using an additive manufacturing, forging, casting, melting, or growing process. In another aspect, the device comprises titanium, tantalum, stainless steel, cobalt chrome molybdenum, or a plastic.

As embodied and broadly described herein, an aspect of the present disclosure relates to a method for additive manufacturing of a spinal interbody device including using additive manufacturing to make the spinal interbody device, including an interbody; and a movable backout prevention mechanism disposed at least partially within the interbody, including a body and one or more lobes integral with the body, wherein each of the one or more lobes is configured to be moved between an unlocked position for insertion of a fixation member and a locked position for preventing backout of the fixation member; wherein the mechanism is additively manufactured with the backout prevention mechanism disposed at least partially within a void formed within a portion of the interbody. In one aspect, the one or more lobes are each configured to be rotatably moved between the unlocked position and the locked position.

### BRIEF DESCRIPTION OF THE DRAWINGS

For a more complete understanding of the features and advantages of the present invention, reference is now made to the detailed description of the invention along with the accompanying figures, in which:

FIG. 1 shows a front view of a spinal interbody device including one or more bodies manufactured together with a rotatably actuated movable backout prevention mechanism in the unlocked position.

FIG. 2 shows a front view of an interbody with the movable backout prevention mechanism in the locked position and one retention feature covering two fixation member holes.

FIG. 3, shows a cross-sectional view through section B-B (as defined in FIGS. 1 and 2) of the interbody and movable member. In this embodiment the actuated movable member is shown with a conical shape, with clearance to the main body of the implant which allows for actuation after manufacturing is completed, and a single member that covers two fixation member holes.

FIG. 4 shows a cross-sectional view through section A-A (as defined in FIGS. 1 and 2) of the backout prevention mechanism in the unlocked position, internally contained within the interbody. FIG. 4 also shows a spherical detent that keeps the backout prevention mechanism from rotating freely.

FIG. 5A shows a top view of the interbody and the backout prevention mechanism.

FIG. 5B shows a cross-sectional view through section C-C(as defined in FIG. 5A) of the movable mechanism in the open position, internally contained within the interbody. FIG. 5B also shows a spherical detent and indents that keeps the movable mechanism from rotating freely.

FIG. 5C shows a top view of the backout prevention device.

FIG. 5D shows a side view of the backout prevention device.

FIG. 6 shows a flowchart of a method embodiment of the invention.

FIG. 7 shows a flowchart of another method embodiment of the invention.

### DETAILED DESCRIPTION OF THE INVENTION

Illustrative embodiments of the system of the present application are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms such as “above,” “below,” “upper,” “lower,” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

The integrated movable mechanism can be manufactured using additive manufacturing (also known as 3D printing), forging, casting, melting, and growing processes.

The parts are anticipated to be manufactured from one material or a combination of implantable materials, such as titanium, tantalum, stainless steel, cobalt chrome molybdenum, or plastics such as PEEK. The material can be the same or different colors to distinguish the different parts.

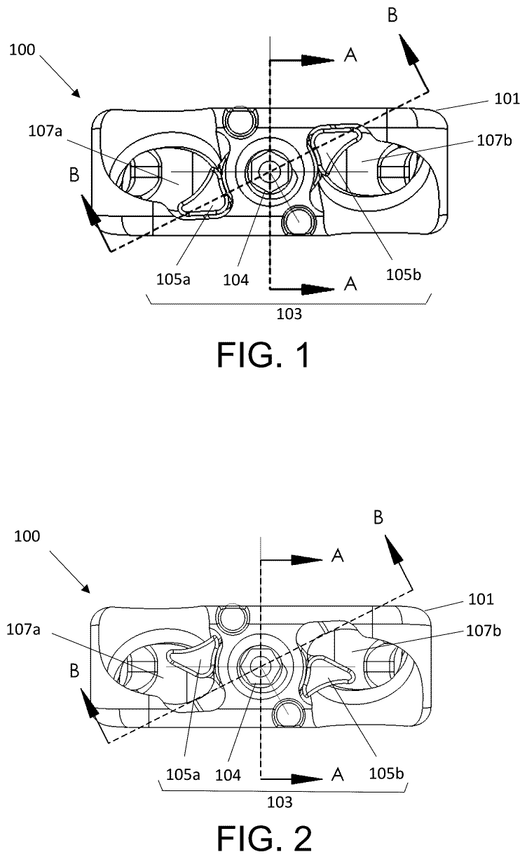

FIGS. 1-5B show one or more aspects of an embodiment of the present invention with particular features as disclosed in detail below. However, embodiments including other features as disclosed herein are encompassed by the present invention.

FIG. 1 shows a front view of an embodiment of a spinal interbody device 100 (also shown in FIGS. 2-5B) that includes an interbody 101 and a movable backout prevention mechanism 103 including a body 104 and two lobes 105a, 105b in the unlocked position, wherein the two lobes 105a, 105b are integral with the body 104. In the unlocked position, the two lobes 105a, 105b each are disposed such that a fixation member such as a screw or anchor can be inserted into each of fixation member holes 107a, 107b to secure the interbody to the vertebra above the interbody 101, the vertebra below the interbody 101, or both. Once fixation members (not shown) have been inserted fully into the fixation member holes 107a, 107b, the backout prevention mechanism 103 can be rotatably actuated to move the lobes 105a, 105b into a locked position, as shown in FIG. 2.

FIG. 2 shows a front view of the interbody 101 with the backout prevention mechanism 103 and its body 104 and lobes 105a, 105b in the locked position, covering the two fixation member holes 107a, 107b. In the locked position, the lobes 105a, 105b partially or completely cover the fixation member holes 107a, 107b, respectively, preventing the fixation members fully inserted into the fixation member holes 107a, 107b from backing out.

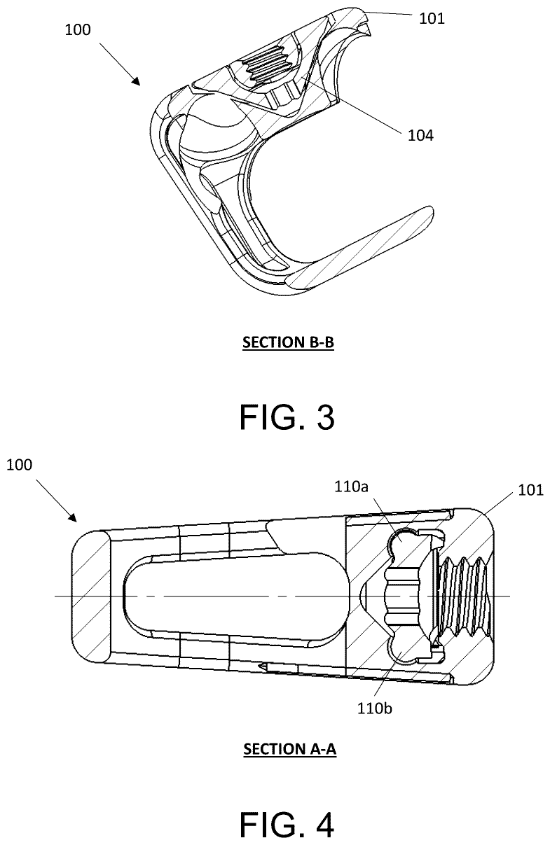

FIG. 3, shows a cross-sectional view of the interbody 101 and the body 104 through section B-B (as defined in FIGS. 1 and 2). In this embodiment the body 104 is shown with a conical shape, with clearance to the main body of the interbody 101 which allows for actuation after manufacturing is completed. The illustrations and discussion herein of a backout prevention mechanism 103 with two lobes 105a, 105b is exemplary and not limiting. It is anticipated that there can be one, two, or more features on the movable mechanism that cover any number of fixation members.

FIG. 4 shows a cross-sectional view of the interbody 101 through section A-A (as defined in FIGS. 1 and 2) of the backout prevention mechanism 103, internally contained within the interbody 101, in the unlocked position. FIG. 4 also shows spherical detents 110a, 110b of the backout prevention mechanism 103 that keep the movable mechanism from rotating freely.

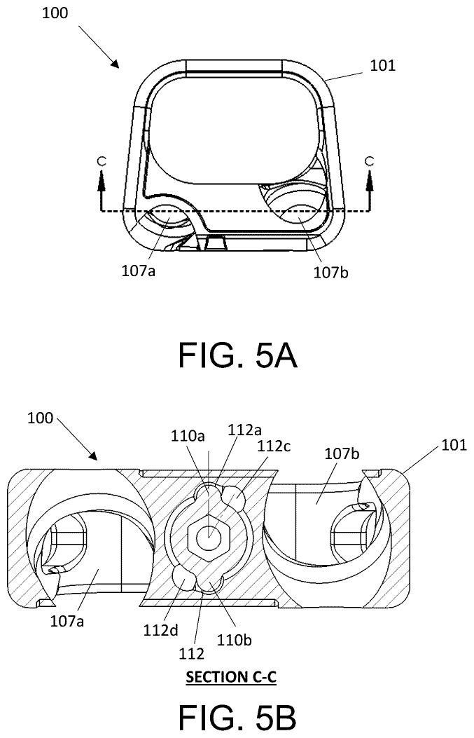

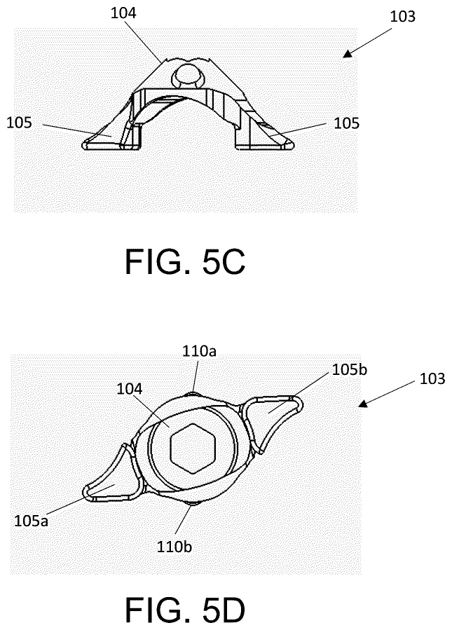

FIG. 5A shows a top view of the interbody 101 with the fixation member holes 107a, 107b and section C-C. FIG. 5B shows a cross-sectional view through section C-C of the backout prevention mechanism 103 in the locked position, internally contained within the interbody 101. FIG. 5B also shows spherical detents 110a, 110b, which keep the backout prevention mechanism 103 from rotating freely when in the locked position. In FIG. 5B, the spherical detents 110a, 110b are shown engaging the indents 112a, 112b, respectively, to prevent free rotation of the backout prevention device while in the locked position. In the open position, the spherical detents 110a, 110b engage the indents 112c, 112d, respectively, to prevent free rotation while in the unlocked position.

FIGS. 5C and 5D show isolated views of the backout prevention mechanism 103, without the surrounding interbody. FIG. 5C shows a top view of the backout prevention mechanism 103, with the body 104 and the lobes 105a, 105b. In this view, the body 104 is shown as conical in shape, but this conical shape is exemplary and non-limiting. In embodiments of the invention, the body 104 is cylindrical, hexagonal, spherical, hemispherical, polyhedral, pyramidal, rectangular, or includes spirally stacked discs. FIG. 5D shows a front view of the backout prevention mechanism 103, with the body 104 and the lobes 105a, 105b.

The movable mechanism can have one or more internal or external features that aid in keeping the movable mechanism lobes 105a, 105b from moving to an undesirable position unless a force or torque is applied. The embodiment shown in FIGS. 1-5B includes two spherical detents 110a, 110b on the backout prevention mechanism 103 that provides tactile feedback and resistance to motion and prevents free rotation of the backout prevention mechanism 103. As shown, the spherical detents are configured to engage the indents 112a-112d (see FIG. 5B). Tactile feedback and rotation resistance can be provided by other features such as interference fits, tapers, clips, or flexures that expand or collapse. Such tactile features may be disposed on the interbody 101 or on the backout prevention mechanism 103, or on another body found on or within the interbody 101, or any combination thereof. Such tactile features can be configured in a variety of shapes including conical, cylindrical, hexagonal, spherical, hemispherical, polyhedral, pyramidal, rectangular, spirally stacked disc-shaped, or any combination thereof, with a number of methods, including the use of additive or subtractive manufacturing methods. Such tactile feature allow the backout prevention mechanism to stay in whatever position is intended by the design.

The embodiment shown in FIGS. 1-5B requires an external force that rotates the backout prevention mechanism 103 from an unlocked to a locked position using a hexagonal drive. It is anticipated that the backout prevention mechanism 103 may be actuated by alternative driving mechanisms such as, e.g., a Philips drive, a hexalobular drive, or a square drive. In embodiments of the present invention, the backout prevention mechanism 103 may be positioned in its resting state, which is the locked position, with a self-actuating spring-loaded mechanism to rotate the backout prevention mechanism 103 to the unlocked position without the use of a drive mechanism, then once the fixation members pass the movable mechanism lobes 105a, 105b, the spring returns the backout prevention mechanism 103 to the locked position. In the embodiment shown in FIGS. 1-5B, the backout prevention mechanism has a rotational motion, but the backout prevention mechanism 103 can be configured to move the lobes 105a, 105b by rotation, sliding, cam, or deflection types of motion.

The embodiment of FIGS. 1-5B has the backout prevention mechanism 103 manufactured between the anterior face of the anterior wall of the interbody 101 and posterior face of the anterior wall of the interbody 101. In other embodiments, the backout prevention mechanism 103 can be manufactured in alternative locations within the interbody. The backout prevention mechanism 103 can be on the medial, lateral, or posterior aspects of the interbody depending on the mode of actuation of the mechanism. The shape of the body 104 of the backout prevention mechanism 103 in the embodiment of FIGS. 1-5B is conical, but a body 104 that is cylindrical, hexagonal, spherical, hemispherical, polyhedral, pyramidal, rectangular, or includes spirally stacked discs, can be used in other embodiments of the present invention. The of the lobes 105a, 105b that cover the fixation members can be leaf-shaped (as shown in the embodiment of FIGS. 1-5D) or can have other shapes suitable for preventing backout of a fixation member.

Embodiments of the present invention include one or more movable backout prevention mechanisms that are manufactured together with the interbody and then disassembled after use, so that only the interbody remains in place. Embodiments can also include alternate instruments such as drill guides, inserters, drivers, and fasteners, which can be manufactured together with the spinal interbody device and detached from the spinal interbody device after implantation. Embodiments also include fixation members that are manufactured together (e.g., by additive manufacturing) with the interbody, and then through an act of user, the fixation members are inserted through the interbody and into the vertebral body. Additionally, embodiments include any combination of interbodies and instruments or other features that are built during the manufacturing process (e.g., additive manufacturing) in a multitude of shapes and combinations of shapes that allow the manufacturing process to continuously print movable backout prevention mechanism.

Embodiments of the present invention include a spinal interbody device kit including the spinal interbody device, which includes an interbody (e.g., interbody 101) and a movable backout prevention mechanism (e.g., backout prevention mechanism 103) disposed at least partially within the interbody, including a body and one or more lobes, wherein each of the one or more lobes is configured to be moved between an unlocked position for insertion of a fixation member and a locked position for preventing backout of the fixation member; and one or more tools to manipulate the backout prevention mechanism.

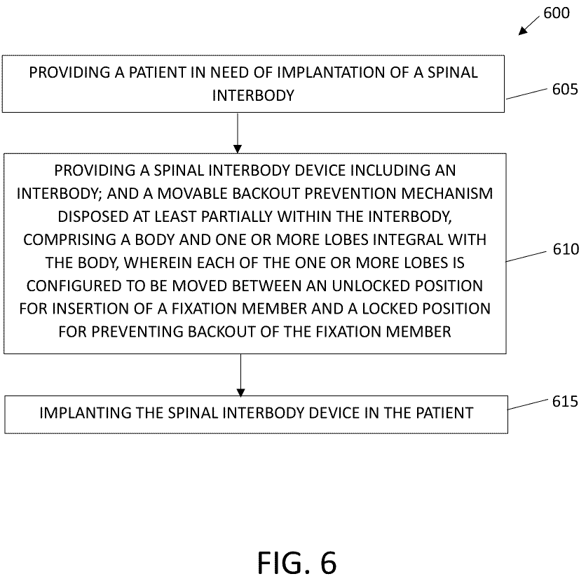

Embodiments of the present invention include a method 600, shown in FIG. 6, of using a spinal interbody device including Block 605, providing a patient in need of implantation of a spinal interbody. Method 600 also includes Block 610, providing a spinal interbody device including an interbody (e.g., interbody 101) and a movable backout prevention mechanism (e.g., backout prevention mechanism 103) disposed at least partially within the interbody, including a body and one or more lobes integral with the body, wherein each of the one or more lobes is configured to be moved between an unlocked position for insertion of a fixation member and a locked position for preventing backout of the fixation member. Method 600 further includes Block 615, implanting the spinal interbody device in the patient.

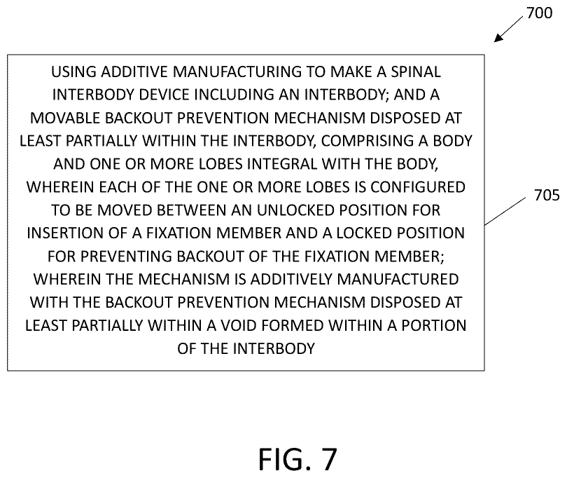

Embodiments of the present invention include a method 700, shown in FIG. 7, for additive manufacturing of a spinal interbody device including Block 705, using additive manufacturing to make a spinal interbody device including an interbody (e.g. interbody 101) and a movable backout prevention mechanism (e.g., backout prevention mechanism 103) disposed within the interbody, including a body and one or more lobes integral with the body, wherein each of the one or more lobes is configured to be moved between an unlocked position for insertion of a fixation member and a locked position for preventing backout of the fixation member, wherein the mechanism is additively manufactured with the backout prevention mechanism disposed within a void formed within a portion of the interbody.

As embodied and broadly described herein, an aspect of the present disclosure relates to a spinal interbody device that comprises, consists essentially of, or consists of an interbody; and a movable backout prevention mechanism disposed at least partially within the interbody, including a body and one or more lobes integral with the body, wherein each of the one or more lobes is configured to be moved between an unlocked position for insertion of a fixation member and a locked position for preventing backout of the fixation member. In one aspect, the backout prevention mechanism is configured to be rotatably moved between the unlocked position and the locked position. In another aspect, the backout prevention mechanism includes a detent configured to engage an indent to provide tactile feedback to a user and to prevent free rotation of the backout prevention mechanism. In another aspect, at least a portion of the backout prevention mechanism is disposed between an anterior face of an anterior wall of the interbody and a posterior face of the anterior wall. In another aspect, the body has a shape that is conical, cylindrical, hexagonal, spherical, hemispherical, polyhedral, pyramidal, rectangular, or leaf-shaped, or includes spirally stacked discs. In another aspect, the device is manufactured using an additive manufacturing, forging, casting, melting, or growing process. In another aspect, the device includes titanium, tantalum, stainless steel, cobalt chrome molybdenum, or a plastic.

As embodied and broadly described herein, an aspect of the present disclosure relates to a spinal interbody device that comprises, consists essentially of, or consists of an interbody; and a movable backout prevention mechanism disposed at least partially within the interbody including a body and one or more lobes integral with the body, wherein each of the one or more lobes is configured to be moved between an unlocked position for insertion of a fixation member and a locked position for preventing backout of the fixation member; and one or more tools to manipulate the backout prevention mechanism. In one aspect, the backout prevention mechanism is configured to be rotatably moved between the unlocked position and the locked position. In another aspect, the backout prevention mechanism includes a detent configured to engage an indent to provide tactile feedback to a user and to prevent free rotation of the backout prevention mechanism. In another aspect, at least a portion of the backout prevention mechanism is disposed between an anterior face of an anterior wall of the interbody and a posterior face of the anterior wall. In another aspect, the body has a shape that is conical, cylindrical, hexagonal, spherical, hemispherical, polyhedral, pyramidal, rectangular, or leaf-shaped, or includes spirally stacked discs. In another aspect, the device is manufactured using an additive manufacturing, forging, casting, melting, or growing process. In another aspect, the device includes titanium, tantalum, stainless steel, cobalt chrome molybdenum, or a plastic.

As embodied and broadly described herein, an aspect of the present disclosure relates to a method of using a spinal interbody device that comprises, consists essentially of, or consists of providing a patient in need of implantation of a spinal interbody; providing the spinal interbody device, including an interbody; and a movable backout prevention mechanism disposed at least partially within the interbody, including a body and one or more lobes integral with the body, wherein each of the one or more lobes is configured to be moved between an unlocked position for insertion of a fixation member and a locked position for preventing backout of the fixation member; and implanting the spinal interbody device in the patient. In one aspect, the one or more lobes are each configured to be rotatably moved between the unlocked position and the locked position. In another aspect, the backout prevention mechanism includes a detent configured to engage an indent to provide tactile feedback to a user and to prevent free rotation of the backout prevention mechanism. In another aspect, at least a portion of the backout prevention mechanism is disposed between an anterior face of an anterior wall of the interbody and a posterior face of the anterior wall. In another aspect, the body has a shape that is conical, cylindrical, hexagonal, spherical, hemispherical, polyhedral, pyramidal, rectangular, or leaf-shaped, or includes spirally stacked discs. In another aspect, the device is manufactured using an additive manufacturing, forging, casting, melting, or growing process. In another aspect, the device comprises titanium, tantalum, stainless steel, cobalt chrome molybdenum, or a plastic.

As embodied and broadly described herein, an aspect of the present disclosure relates to a method for additive manufacturing of a spinal interbody device that comprises, consists essentially of, or consists of using additive manufacturing to make the spinal interbody device, including an interbody; and a movable backout prevention mechanism disposed at least partially within the interbody, including a body and one or more lobes integral with the body, wherein each of the one or more lobes is configured to be moved between an unlocked position for insertion of a fixation member and a locked position for preventing backout of the fixation member; wherein the mechanism is additively manufactured with the backout prevention mechanism disposed at least partially within a void formed within a portion of the interbody. In one aspect, the one or more lobes are each configured to be rotatably moved between the unlocked position and the unlocked position.

While various features of the present invention are discussed and illustrated separately for clarity, each and every feature disclosed herein can be combined with any other disclosed feature in an embodiment unless such a combination is physically impossible.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of.” As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step, or a limitation) or group of integers (e.g., feature(s), element(s), characteristic(s), property(ies), method/process(s) steps, or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the devices and/or methods disclosed and claimed herein can be made and executed without undue experimentation in light of the present disclosure. While the devices and/or methods of this invention have been described in terms of particular embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope, and concept of the invention as defined by the appended claims.

Furthermore, no limitations are intended to the details of construction or design herein shown, other than as described in the claims below. It is therefore evident that the particular embodiments disclosed above may be altered or modified and all such variations are considered within the scope and spirit of the disclosure. Accordingly, the protection sought herein is as set forth in the claims below.

Modifications, additions, or omissions may be made to the systems and apparatuses described herein without departing from the scope of the invention. The components of the systems and apparatuses may be integrated or separated. Moreover, the operations of the systems and apparatuses may be performed by more, fewer, or other components. The methods may include more, fewer, or other steps. Additionally, steps may be performed in any suitable order.

To aid the Patent Office, and any readers of any patent issued on this application in interpreting the claims appended hereto, applicants wish to note that they do not intend any of the appended claims to invoke 35 U.S.C. § 112 (f) as it exists on the date of filing hereof unless the words “means for” or “step for” are explicitly used in the particular claim.

What is claimed is:

1. A spinal interbody device comprising:
an interbody; and
a movable backout prevention mechanism disposed at least partially within the interbody, comprising a body and one or more lobes integral with the body, wherein each of the one or more lobes is configured to be moved between an unlocked position for insertion of a fixation member and a locked position for preventing backout of the fixation member.

2. The device of claim 1, wherein the backout prevention mechanism is configured to be rotatably moved between the unlocked position and the locked position.

3. The device of claim 1, wherein the backout prevention mechanism comprises a detent configured to engage an indent to provide tactile feedback to a user and to prevent free rotation of the backout prevention mechanism.

4. The device of claim 1, wherein at least a portion of the backout prevention mechanism is disposed between an anterior face of an anterior wall of the interbody and a posterior face of the anterior wall.

5. The device of claim 1, wherein the body has a shape that is conical, cylindrical, hexagonal, spherical, hemispherical, polyhedral, pyramidal, rectangular, or leaf-shaped, or comprises spirally stacked discs.

6. The device of claim 1, wherein the device is manufactured using an additive manufacturing, forging, casting, melting, or growing process.

7. The device of claim 1, wherein the device comprises titanium, tantalum, stainless steel, cobalt chrome molybdenum, or a plastic.

8. A spinal interbody device kit comprising:
a spinal interbody device comprising:
an interbody; and
a movable backout prevention mechanism disposed at least partially within the interbody, comprising a body and one or more lobes integral with the body, wherein each of the one or more lobes is configured to be moved between an unlocked position for insertion of a fixation member and a locked position for preventing backout of the fixation member; and
one or more tools to manipulate the backout prevention mechanism.

9. The kit of claim 8, wherein the backout prevention mechanism is configured to be rotatably moved between the unlocked position and the locked position.

10. The kit of claim 8, wherein the backout prevention mechanism comprises a detent configured to engage an indent to provide tactile feedback to a user and to prevent free rotation of the backout prevention mechanism.

11. The kit of claim 8, wherein at least a portion of the backout prevention mechanism is disposed between an anterior face of an anterior wall of the interbody and a posterior face of the anterior wall.

12. The kit of claim 8, wherein the body has a shape that is conical, cylindrical, hexagonal, spherical, hemispherical, polyhedral, pyramidal, rectangular, or leaf-shaped, or comprises spirally stacked discs.

13. The kit of claim 8, wherein the device is manufactured using an additive manufacturing, forging, casting, melting, or growing process.

14. The kit of claim 8, wherein the device comprises titanium, tantalum, stainless steel, cobalt chrome molybdenum, or a plastic.

15. A method of using a spinal interbody device comprising:
providing a patient in need of implantation of a spinal interbody;
providing the spinal interbody device, comprising:
an interbody; and
a movable backout prevention mechanism disposed at least partially within the interbody, comprising a body and one or more lobes integral with the body, wherein each of the one or more lobes is configured to be moved between an unlocked position for insertion of a fixation member and a locked position for preventing backout of the fixation member; and
implanting the spinal interbody device in the patient.

16. The method of claim 15, wherein the one or more lobes are each configured to be rotatably moved between the unlocked position and the locked position.

17. The method of claim 15, wherein the backout prevention mechanism comprises a detent configured to engage an indent to provide tactile feedback to a user and to prevent free rotation of the backout prevention mechanism.

18. The method of claim 15, wherein at least a portion of the backout prevention mechanism is disposed between an anterior face of an anterior wall of the interbody and a posterior face of the anterior wall.

19. The method of claim 15, wherein the body has a shape that is conical, cylindrical, hexagonal, spherical, hemispherical, polyhedral, pyramidal, rectangular, or leaf-shaped, or comprises spirally stacked discs.

20. The method of claim 15, wherein the device is manufactured using an additive manufacturing, forging, casting, melting, or growing process.

21. The method of claim 15, wherein the device comprises titanium, tantalum, stainless steel, cobalt chrome molybdenum, or a plastic.

22. A method for additive manufacturing of a spinal interbody device comprising:
using additive manufacturing to make the spinal interbody device, comprising:
an interbody; and
a movable backout prevention mechanism disposed at least partially within the interbody, comprising a body and one or more lobes integral with the body, wherein each of the one or more lobes is configured to be moved between an unlocked position for insertion of a fixation member and a locked position for preventing backout of the fixation member;
wherein the mechanism is additively manufactured with the backout prevention mechanism disposed at least partially within a void formed within a portion of the interbody.

23. The method of claim 22, wherein the one or more lobes are each configured to be rotatably moved between the unlocked position and the unlocked position.

24. The method of claim 22, wherein the backout prevention mechanism comprises a detent configured to engage an indent to provide tactile feedback to a user and to prevent free rotation of the backout prevention mechanism.

25. The method of claim 22, wherein the at least a portion of the backout prevention mechanism is disposed between an anterior face of an anterior wall of the interbody and a posterior face of the anterior wall.

26. The method of claim 22, wherein the body has a shape that is conical, cylindrical, hexagonal, spherical, hemispherical, polyhedral, pyramidal, rectangular, or leaf-shaped, or comprises spirally stacked discs.

27. The method of claim 22, wherein the device comprises titanium, tantalum, stainless steel, cobalt chrome molybdenum, or a plastic.
